(19) United States
(12) Patent Application Publication
(10) Pub. No.: US 2023/0395199 A1
(43) Pub. Date: Dec. 7, 2023

(54) ARTIFICIAL PHOTOSYNTHESIS OPTIMIZATION

(71) Applicant: INTERNATIONAL BUSINESS MACHINES CORPORATION, ARMONK, NY (US)

(72) Inventors: Sarbajit K. Rakshit, Kolkata (IN); Jagabondhu Hazra, BANGALORE (IN); Manikandan Padmanaban, Chennai (IN)

(21) Appl. No.: 17/805,241

(22) Filed: Jun. 3, 2022

(51) Int. Cl. G16B 99/00; G06F 1/03; B01J 35/00

(52) U.S. Cl. CPC G16B 99/00; B01J 35/004; G06F 1/03

(57) ABSTRACT

A method for photosynthesis optimization including determining ambient levels of at least one gas, water, and sunlight at a location. A catalyst is selected to perform an artificial photosynthesis reaction at the location. At least one limiting factor is determined for the artificial photosynthesis reaction based on the catalyst and the ambient levels, and the at least one limiting factor is compensated for.

### BACKGROUND

Exemplary embodiments of the present inventive concept relate to artificial photosynthesis, and more particularly, to artificial photosynthesis optimization.

Artificial photosynthesis is a chemical process that mimics natural photosynthesis principles with up to a 14-fold greater efficiency. Artificial photosynthesis reactions may reduce anthropogenic emissions of carbon dioxide (CO₂) and produce useful by-products (e.g., fuel, food, chemicals (e.g., organic compounds), plastics, etc.) in the process. In artificial photosynthesis reactions, a catalyst (e.g., an artificial leaf, a semiconductor, etc.) must be able to use sunlight and water (H₂O) to reduce CO₂ and H₂O into H₂.

However, the availability of CO₂, H₂O and sunlight is often not present with required/optimal proportions at the location of an artificial photosynthesis reaction. For example, levels of sunlight, CO₂ and humidity can fluctuate widely depending on numerous conditions, such as a location, atmospheric pollutants, season, weather, time of day, etc. A big challenge with artificial photosynthesis reactions is getting a catalyst's sunlight-charged particles to persist long enough to perform the chemical reactions for utilisation. The catalyst's charged particles separate when the sunlight's energy is absorbed, but they can also come back together very quickly. There is a need to control humidity and sunlight at the location of an artificial photosynthesis reaction to keep all the charged particles engaged completely in the chemical reaction for maximum utilization, and to capture CO₂ to the fullest extent possible. However, excessive or sustained sunlight can also damage a catalyst. Furthermore, optimal input levels may depend on a type of catalyst being used and a cost-benefit-analysis of adjusting inputs. To provide the greatest efficiency for artificial photosynthesis reactions, consideration of these variables must be carefully performed.

### SUMMARY

Exemplary embodiments of the present inventive concept relate to a method, a computer program product, and a system for providing photosynthesis optimization.

According to an exemplary embodiment of the present inventive concept, a method may be provided for photosynthesis optimization including determining ambient levels of at least one gas, water, and sunlight at a location. A catalyst is selected to perform an artificial photosynthesis reaction at the location. At least one limiting factor is determined for the artificial photosynthesis reaction based on the catalyst and the ambient levels, and the at least one limiting factor is compensated for.

According to an exemplary embodiment of the present inventive concept, a computer program product may be provided for photosynthesis optimization. The computer program product may include one or more non-transitory computer-readable storage media and program instructions stored on the one or more non-transitory computer-readable storage media capable of performing a method. The method includes determining ambient levels of at least one gas, water, and sunlight at a location. A catalyst is selected to perform an artificial photosynthesis reaction at the location. At least one limiting factor for the artificial photosynthesis reaction is determined based on the catalyst and the ambient levels, and the at least one limiting factor is compensated for.

According to an exemplary embodiment of the present inventive concept, a computer system for photosynthesis optimization may be provided. The system includes one or more computer processors, one or more computer-readable storage media, and program instructions stored on the one or more of the computer-readable storage media for execution by at least one of the one or more processors capable of performing a method. The method includes determining ambient levels of at least one gas, water, and sunlight at a location. A catalyst is selected to perform an artificial photosynthesis reaction at the location. At least one limiting factor for the artificial photosynthesis reaction is determined based on the catalyst and the ambient levels, and the at least one limiting factor is compensated for.

### BRIEF DESCRIPTION OF THE DRAWINGS

The following detailed description, given by way of example and not intended to limit the exemplary embodiments solely thereto, will best be appreciated in conjunction with the accompanying drawings, in which:

FIG. 1 illustrates a schematic diagram of a photosynthesis optimization system 100, in accordance with an exemplary embodiment of the present inventive concept.

FIG. 2A illustrates a flowchart of photosynthesis optimization 200 provided by a photosynthesis optimization program 134 of the photosynthesis optimization system 100, in accordance with an exemplary embodiment of the present inventive concept.

FIG. 2B illustrates a catalyst 3D lookup table used by the photosynthesis optimization program 134 to implement the photosynthesis optimization 200 of FIG. 2A, according to an exemplary embodiment of the present inventive concept.

FIG. 2C illustrates an exemplary methodology of prediction horizon optimization used by the photosynthesis optimization program 134 to implement the photosynthesis optimization 200 of FIG. 2A.

FIG. 2D illustrates an exemplary system used by the photosynthesis optimization program 134 to implement the photosynthesis optimization 200 of FIG. 2A.

FIG. 3 illustrates a block diagram depicting the hardware components included in the photosynthesis optimization system 100 of FIG. 1, in accordance with an exemplary embodiment of the present inventive concept.

FIG. 4 illustrates a cloud computing environment, in accordance with an exemplary embodiment of the present inventive concept.

FIG. 5 illustrates abstraction model layers, in accordance with an exemplary embodiment of the present inventive concept.

It is to be understood that the included drawings are not necessarily drawn to scale/proportion. The included drawings are merely schematic examples to assist in understanding of the present inventive concept and are not intended to portray fixed parameters. In the drawings, like numbering may represent like elements.

### DETAILED DESCRIPTION OF THE DRAWINGS

Exemplary embodiments of the present inventive concept are disclosed hereafter. However, it shall be understood that the scope of the present inventive concept is not limited thereto. The disclosed exemplary embodiments are merely illustrative of the claimed system, method, and computer program product. The present inventive concept may be embodied in many different forms and should not be construed as limited to only the exemplary embodiments set forth herein. Rather, these included exemplary embodiments are provided for completeness of disclosure and to facilitate an understanding to those skilled in the art. In the detailed description, discussion of well-known features and techniques may be omitted to avoid unnecessarily obscuring the presented exemplary embodiments.

References in the specification to “one embodiment,” “an embodiment,” “an exemplary embodiment,” etc., indicate that the embodiment described may include a particular feature, structure, or characteristic, but not every embodiment may necessarily include that feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to implement such feature, structure, or characteristic in connection with other embodiments whether or not explicitly described.

In the interest of not obscuring the presentation of the exemplary embodiments of the present inventive concept, in the following detailed description, some processing steps or operations that are known in the art may have been combined for presentation and for illustration purposes, and in some instances, may have not been described in detail. Additionally, some processing steps or operations that are known in the art may not be described at all. The following detailed description is focused on the distinctive features or elements of the present inventive concept according to various exemplary embodiments.

As previously mentioned, efficient artificial photosynthesis reactions depend on, for example, optimal levels of ambient CO₂, H₂O, and sunlight. These input levels can vary according to the location, atmospheric pollutants, season, weather, time of day, catalyst type, cost-benefit-analysis, etc. A human being is incapable of computing all these variables simultaneously with any degree of expediency or accuracy, and even less so with calculating the logistics of adjustments. The present inventive concept enables artificial photosynthesis optimization based on analysis of these variables to provide higher product yields, reduced expense, and reduced diversion of human resources.

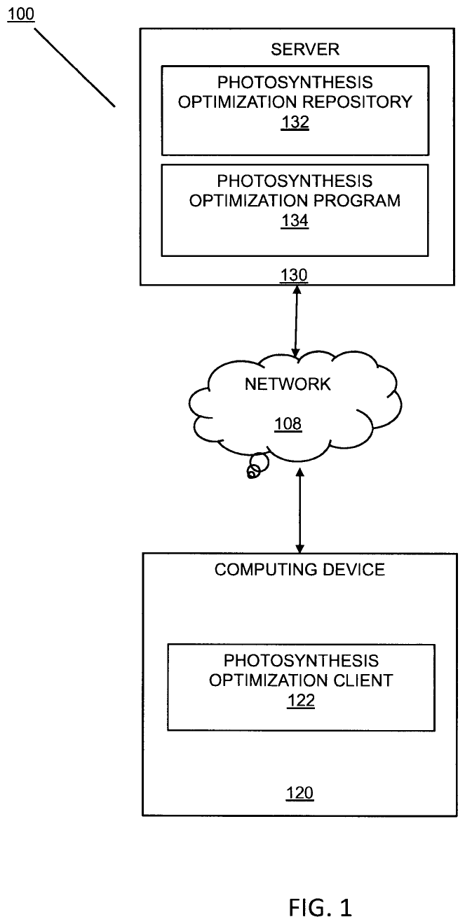

FIG. 1 depicts a schematic diagram of the photosynthesis optimization system 100, in accordance with an exemplary embodiment of the present inventive concept.

The photosynthesis optimization system 100 may include a user-operated computing device 120 and a photosynthesis optimization server 130, which may all be interconnected via a network 108. Programming and data content may be stored and accessed remotely across several servers via the network 108. Alternatively, programming and data may be stored locally on as few as one physical computing device 120 or stored amongst multiple computing devices.

According to the exemplary embodiment of the present inventive concept depicted in FIG. 1, the network 108 may be a communication channel capable of transferring data between connected devices. The network 108 may be the Internet, representing a worldwide collection of networks 108 and gateways to support communications between devices connected to the Internet. Moreover, the network 108 may utilize various types of connections such as wired, wireless, fiber optic, etc., which may be implemented as an intranet network, a local area network (LAN), a wide area network (WAN), or a combination thereof. The network 108 may be a Bluetooth network, a Wi-Fi network, or a combination thereof. The network 108 may operate in frequencies including 2.4 GHz and 5 GHz internet, near-field communication, Z-Wave, Zigbee, etc. The network 108 may be a telecommunications network used to facilitate telephone calls between two or more parties comprising a landline network, a wireless network, a closed network, a satellite network, or a combination thereof. In general, the network 108 may represent any combination of connections and protocols that will support communications between connected devices.

The computing device 120 may include a photosynthesis optimization client 122, and may be an enterprise server, a laptop computer, a notebook, a tablet computer, a netbook computer, a personal computer (PC), a desktop computer, a server, a personal digital assistant (PDA), a smart phone, a mobile phone, a virtual device, a thin client, an IoT device, or any other electronic device or computing system capable of sending and receiving data to and from other computing devices. The computing device 120 may be connected to various measuring devices, such as for measuring ambient levels of gases, humidity, and/or light. Although the computing device 120 is shown as a single device, the computing device 120 may be comprised of a cluster or plurality of computing devices, in a modular manner, etc., working together or working independently.

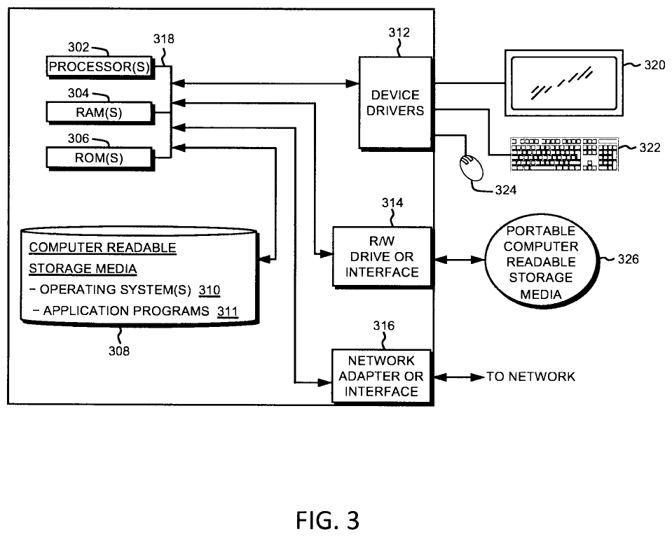

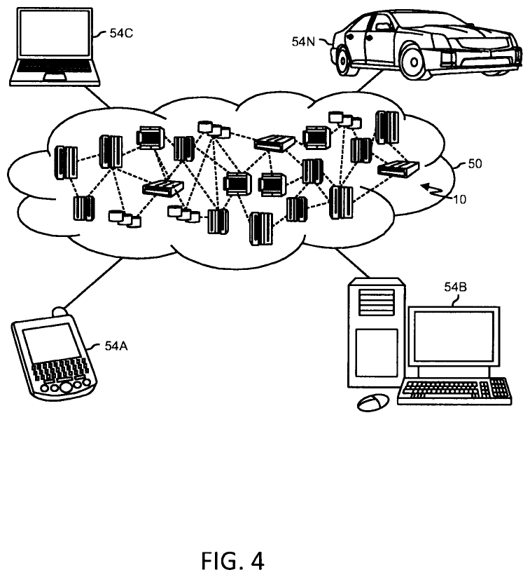

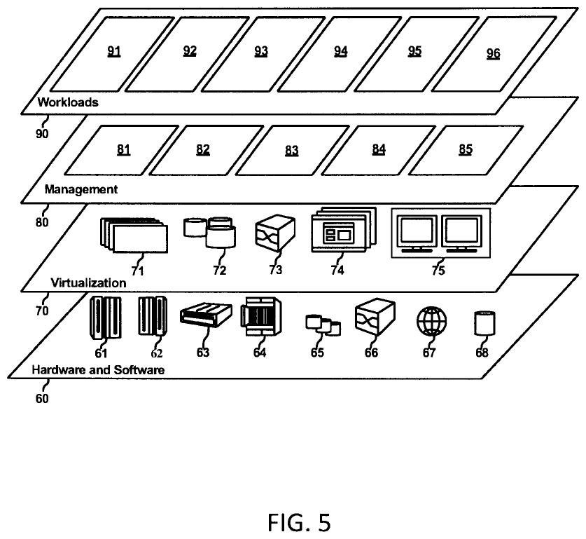

The computing device 120 is described in greater detail as a hardware implementation with reference to FIG. 3, as part of a cloud implementation with reference to FIG. 4, and/or as utilizing functional abstraction layers for processing with reference to FIG. 5.

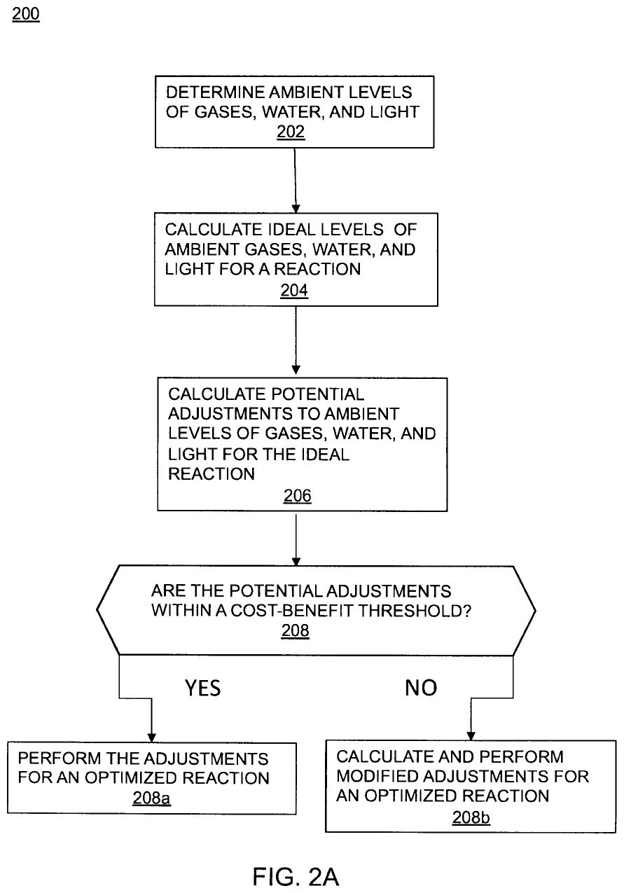

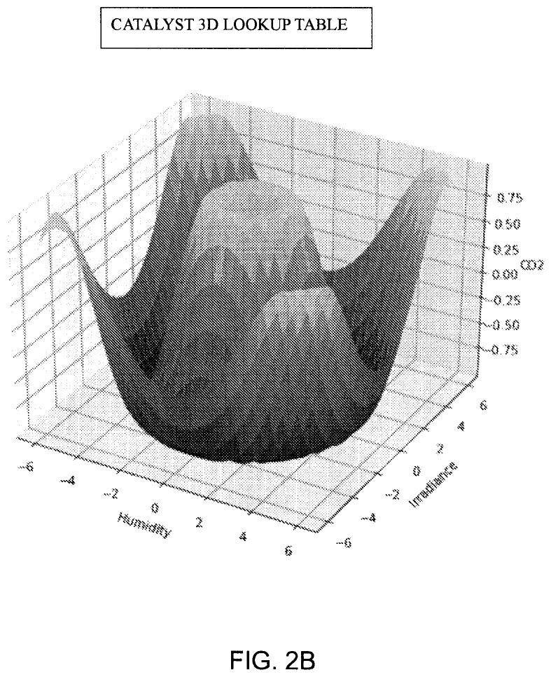

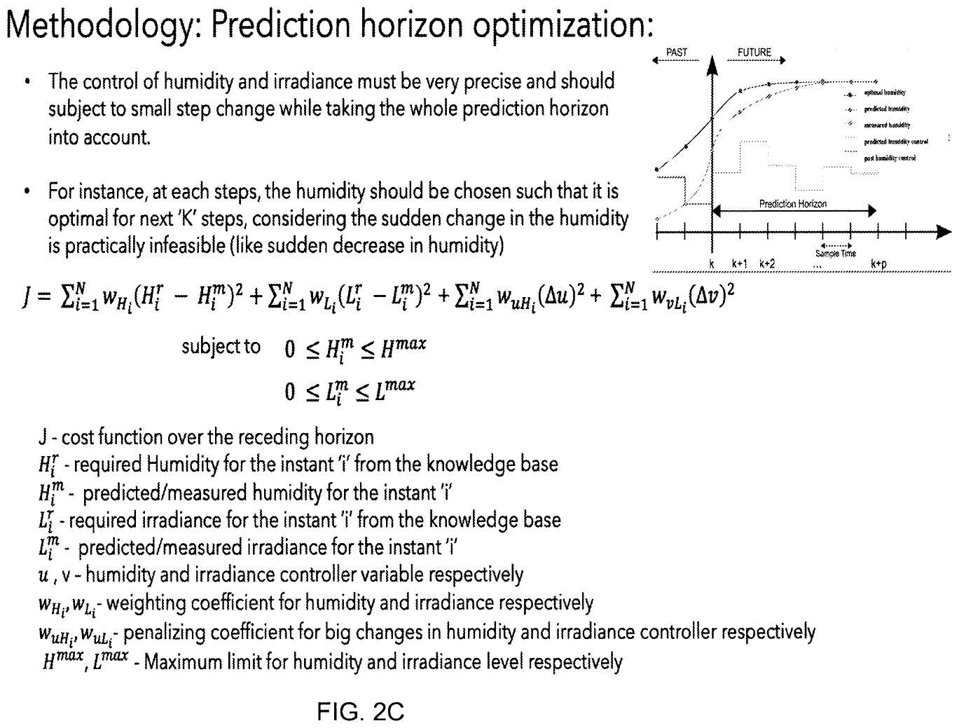

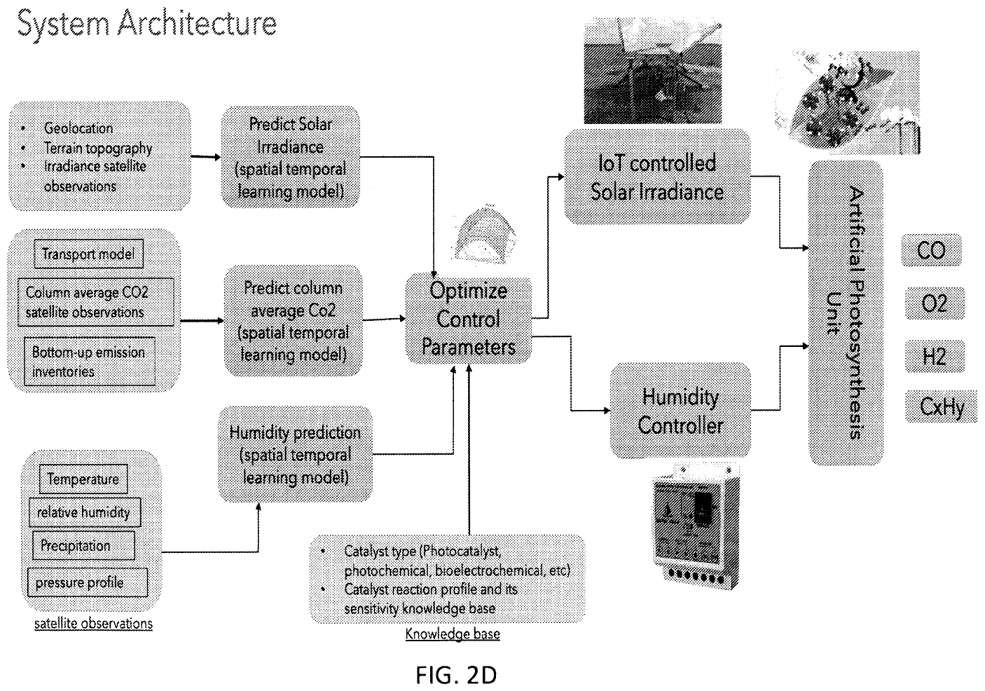

The photosynthesis optimization client 122 may act as a client in a client-server relationship with a server, for example the photosynthesis optimization server 130. The photosynthesis optimization client 122 may be a software and/or a hardware application capable of communicating with and providing a user interface for a user to interact with the photosynthesis optimization server 130 and/or other computing devices via the network 108. Moreover, the photosynthesis optimization client 122 may be capable of transferring data between the computing device 120 and other computer devices/servers via the network 108. The photosynthesis optimization client 122 may utilize various wired and wireless connection protocols for data transmission and exchange, including Bluetooth, 2.4 GHz and 5 GHz internet, near-field communication, etc. The photosynthesis optimization client 122 is described in greater detail with respect to FIGS. 2-5.

The photosynthesis optimization server 130 may include a photosynthesis optimization repository 132 for storing various data (described hereinafter) and the photosynthesis optimization program 134 (also described hereinafter). The photosynthesis optimization server 130 may act as a server in a client-server relationship with a client, e.g., the photosynthesis optimization client 122. The photosynthesis optimization server 130 may be an enterprise server, a laptop computer, a notebook, a tablet computer, a netbook computer, a personal computer (PC), a desktop computer, a server, a personal digital assistant (PDA), a rotary phone, a touchtone phone, a smart phone, a mobile phone, a virtual device, a thin client, an IoT device, or any other electronic device or computing system capable of sending and receiving data to and from other computing devices. Although the photosynthesis optimization server 130 is shown as a single computing device, the present inventive concept is not limited thereto. For example, the photosynthesis optimization server 130 may be comprised of a cluster or plurality of computing devices, in a modular manner, etc., working together or working independently.

The photosynthesis optimization server 130 is described in greater detail as a hardware implementation with reference to FIG. 3, as part of a cloud implementation with reference to FIG. 4, and/or as utilizing functional abstraction layers for processing with reference to FIG. 5. The photosynthesis optimization program 134 and/or the photosynthesis optimization client 122 may be software and/or hardware programs that may facilitate photosynthesis optimization discussed in further detail with reference to FIGS. 2-5.

FIG. 2A illustrates the flowchart of photosynthesis optimization 200, in accordance with an exemplary embodiment of the present inventive concept.

The photosynthesis optimization program 134 may determine ambient levels of gases, water, and light (step 202). The ambient levels of gases (e.g., CO₂, oxygen (O₂), pollutants, etc.), water (e.g., humidity), and light (e.g., sunlight) may be dynamically measured and/or predicted at a predetermined location. Ambient levels may refer to quantified measurements of gas (e.g., partial pressures), water (e.g., humidity), and light (e.g., irradiance intensity). The location may host or be a prospective host for a catalyst (e.g., an artificial photosynthesis substrate and/or a photosynthetic organism).

The location may be an area with predetermined boundaries (e.g., a geolocation, region, town/city, property, activity site, an enclosed space, etc.). A geolocation may refer to a location based on a specific longitude and latitude. A property may refer to a location based on dimensions of real property. An activity site may refer to a location of anthropogenic gaseous emission (e.g., CO₂) production, such as an industrial complex or part thereof (e.g., a smokestack). An enclosed space may refer to a location with tangible boundaries (e.g., a walled structure, an artificial photosynthesis unit, a greenhouse, etc.). The location may be selected by the user or the photosynthesis optimization program 134. The photosynthesis optimization program 134 may select the location based on a catalyst and/or measured or predicted ambient levels of gases, water, and light. Similarly, the photosynthesis optimization program 134 may select a catalyst based on a location and/or measured or predicted ambient levels of gases, water, and light.

The photosynthesis optimization program 134 may obtain ambient levels of the gases, water, and light at the location via the network 108 from a local computing device 120 and/or the internet. Multimedia may be obtained related to the location (e.g., satellite imaging (e.g., irradiance and column average CO₂), bottom-up emission inventories, terrain topography, location dimensions, weather forecasts (e.g., temperature, relative humidity, precipitation, pressure profile), industrial project details, almanacs, and/or published ambient levels of gases, water, and light, etc. The photosynthesis optimization program 134 may perform machine learning (e.g., natural language processing (NLP), optical character recognition (OCR), etc.) and extract relevant location features and/or ambient levels of gases, water, and light. A spatial temporal learning model may be generated to predict solar irradiance, column CO₂ averages, and humidity at the location based on correlation with corroborating measurements and/or extracted features.

For example, the photosynthesis optimization program 134 may measure the ambient levels of CO₂, humidity, and sunlight at a factory premises using a computing device 120 thereat. The ambient level measurements taken may be correlated with weather forecasts and/or satellite imaging. A spatial temporal model may learn ambient levels of CO₂, humidity, and sunlight at the same location, and predict ambient levels at other locations with similar characteristics. Satellite imaging analysis of a nearby stretch of highway reveals comparable ambient levels of CO₂, humidity, and sunlight.

The photosynthesis optimization program 134 may calculate ideal levels of ambient gases, water, and light for a reaction (step 204). The ideal levels of gasses, water, and light may refer to peak proportions/concentrations calculated for a photosynthetic reaction (natural and/or artificial) as performed by at least one catalyst at the location. In an embodiment, with reference to FIG. 2B, a methodology of prediction horizon optimization may be used. Predicted and/or dynamic ideal levels of gases, water, and light may vary by time (e.g., hour, day, season, etc.). For example, there may be increased ambient CO₂ emissions during rush hour and comparatively less on weekends, etc. Thus, ideal levels of gases, water, and light might not be static at the location. Different segments of time of predetermined lengths may have different predicted and/or dynamic ideal levels of gases, water, and light. The predetermined length of time may itself be variable (e.g., daytime, or for the duration of a condition). The type of catalyst may include a plant, photocatalyst, photochemical catalyst, bio-electrochemical catalyst, etc. An enclosed catalyst (e.g., a controlled environment) may refer to an artificial photosynthesis unit. In a cooperative network of catalysts and/or artificial photosynthesis units, catalyst types may be mixed. The catalyst may be selected based on the ambient levels of gases, water, and light at the location, reaction products produced (e.g., CO, O₂, H₂, CxHy, etc.), and/or the location. The photosynthesis optimization program 134 may select a catalyst (e.g., for the location) based on a sensitivity profile published by the manufacturer which includes the limitations and/or ideal ambient levels of gases, water, and light. For example, the sensitivity profile for the catalyst may include the impact of ambient levels of gases, humidity, and irradiance on CO₂ sequestration, longevity, and/or reaction performance. Each type of catalyst may have a unique sensitivity profile. The sensitivity profile may be analyzed by machine learning processes (e.g., NLP and/or OCR). In an embodiment, the sensitivity profile may also include temperature tolerance. The photosynthesis optimization program 134 may generate a three-dimensional (3D) lookup table for each catalyst that graphs the catalyst's sensitivity profile. In an embodiment, catalyst dimensions, quantity, and/or estimated wear-and-tear may be accounted for in the 3D lookup. The catalyst type; reaction products; ideal ambient levels of gases, water, and light; sensitivity profile; catalyst 3D lookup table; manufacturer multimedia; and measured ambient levels of gases, water, and light at the location may be stored in the photosynthesis optimization repository 132.

For example, with reference to FIG. 2B, the photosynthesis optimization program 134 may select a catalyst for inclusion in a network of artificial photosynthesis units based on the referenced 3D lookup table which permits an ideal artificial photosynthesis reaction given the ambient levels of CO₂, humidity, and sunlight at the factory premises.

The photosynthesis optimization program 134 may calculate potential adjustments to ambient levels of gases, water, and light for the ideal reaction (step 206). The ideal reaction may be an ideal photosynthetic reaction (artificial and/or natural) for at least one selected catalyst and/or location. If multiple different catalysts are involved with unique ideal reactions, the artificial photosynthesis program 134 determine an optimized compromise. The photosynthesis optimization program 134 may compare ideal ambient levels for the ideal reaction with the ambient levels at the location and detect a limiting factor. A limiting factor may refer to a sub-ideal catalyst, proportion/ambient level of gases, water, and/or light that hinders the ideal reaction. The photosynthesis optimization program 134 may refer to the spatial temporal model (if already generated). The inputs to the spatial temporal model may include the levels of ambient gases, water, and light, location data (e.g., climate, day, time, season, etc.), the selected catalyst, etc. Based on the spatial temporal model, the ideal reaction and potential adjustments may be retrieved from the artificial photosynthesis repository 132. An illumination control system and/or an artificial light source may be used to adjust light irradiance to at least a portion of the at least one catalyst. The photosynthesis optimization program 134 may use IoT feed to determine the catalyst's irradiance level and distribution thereof. The photosynthesis optimization program 134 may virtually partition the catalyst into sections and analyse irradiance accordingly. The illumination control system may include lenses and/or reflectors. The lenses and/or reflectors may be connected to inclinometers which adjust the angle and/or position of the lenses and/or reflectors in response to actuators operated by a controller connected to the photosynthesis optimization program 134. The photosynthesis optimization program 134 may perform calculations to increase irradiance (e.g., angles of light incidence, angles of light reflection, lens angles, etc.) to at least a part of the catalyst (e.g., a section with poor irradiance). The controller may be connected to a battery, processor, and an emergency switch. A humidity controller (e.g., a humidifier) may be used to adjust ambient levels of water. The photosynthesis optimization program 134 may determine the amount of water needed to produce the desired humidity (e.g., based on the volume/pressure of the ambient levels of gases and/or the area of an enclosed location, rate of atmospheric dispersal (if open), temperature, etc.). In a network of physically connected artificial photosynthesis units, potential adjustments may include diverting gases (e.g., CO₂), humidity, and/or sunlight from one artificial photosynthesis unit(s) to another, such as by interconnected pipes, vents, and/or valves.

For example, the photosynthesis optimization program 134 may detect decreased levels of CO₂ emanating from the factory in the IoT feed and being sequestered by one of the artificial photosynthesis units. Analysis of a press release indicates that the factory is down for maintenance and thus operations are halted. Fortunately, the other artificial photosynthesis unit has an excess of ambient CO₂ in its vicinity. The photosynthesis optimization program 134 determines that CO₂ can be diverted from the other artificial photosynthesis unit to another artificial photosynthesis unit in the network via pipes to continue an ideal reaction. However, the IoT feed detects decreased irradiance too. Analysis of local weather forecasts indicates that the factory premises will be partly cloudy for the remainder of the day. The photosynthesis optimization program 134 calculates that artificial sunlight will be necessary to preserve the ideal reaction and that adjusting the illumination control system alone will be insufficient.

The photosynthesis optimization program 134 may determine if the potential adjustments are within a cost-benefit threshold (decision 208). The photosynthesis optimization program 134 may perform a cost-benefit analysis before implementing the potential adjustments for an optimized reaction. The photosynthesis optimization program 134 may calculate the ideal reaction's potential adjustment costs (e.g., financial and opportunity costs, etc.) and benefits (e.g., quantified pollutant reduction or oxygen increase, net profit, product yield, value of “green company” recognition, etc.). A threshold for cost expenditure and/or minimum benefit (cost-benefit) may be predetermined (e.g., by the user). Financial resource costs may refer to the monetary cost involved with implementing potential adjustments, such as labour and the predicted task time (e.g., dollars per minute); expenses related to adjusting gases, water, and/or light; and/or fuel and/or electricity expenses to operate, transport, and/or rent equipment for the potential adjustments (e.g., the humidity controller, the illumination control system, etc.). The photosynthesis optimization program 134 may access an enterprise server with employee wages/job responsibilities/titles and/or equipment/material inventory databases (e.g., the enterprise, supplier, rental company, etc.) over the network 108. The photosynthesis optimization program 134 may determine deficiencies and/or costs of potential adjustments. Opportunity costs may include the foregone benefit of diverting a resource from one task to another, such as depleting/diverting supply inventory, reallocating equipment/persons from another task and/or location, and the cost of reduced longevity of equipment and/or the selected catalyst. If the cost of the potential adjustments for the optimized reaction exceeds the predetermined threshold and/or falls short of a minimum benefit, the photosynthesis optimization program 134 may present the discrepancy for the user's approval/rejection. A plurality of cost-benefit compliant potential adjustment options with different cost-benefit values may be displayed to the user or may be selected automatically based on the nearest match. The cost-benefit values may refer to an aggregate score or individual cost and benefit scores. Time segments with different potential adjustments may have different associated cost-benefit values and/or thresholds. In an embodiment, a predetermined cost-benefit buffer zone may be used to allow for unanticipated expenses and delays to the optimized reaction. If the user overrides the optimized reaction, the photosynthesis optimization client 134 may learn accordingly. A cost-benefit analysis model component may be included in the spatial temporal model.

If the answer to decision 208 is “YES”, the photosynthesis optimization program 134 may proceed to step 208a and perform the adjustments for an optimized reaction. The optimized reaction may refer to a reaction that maximizes the cost-benefit value within the predetermined cost-benefit threshold. Thus, the optimized reaction may or may not be the same as the ideal reaction. The photosynthesis optimization program 134 may coordinate logistics of implementing the optimized reaction. For example, the photosynthesis optimization program 134 may provide instructions, scheduling, and/or dispatch humans, remotely operated equipment, and/or machines (e.g., drones) to implement the adjustments and/or the modified adjustments (described below) for the optimized reaction. The spatial temporal model may learn from the context and efficacy of implemented adjustments and/or modified adjustments.

If the answer to decision 208 is “NO”, the photosynthesis optimization program 134 may proceed to step 208b and calculate and perform modified adjustments for an optimized reaction including cost-benefit value parameters. The modified adjustments may include altering at least one of the location, the catalyst(s), use of resources (e.g., equipment, machines, employees, etc.), time of operation, and quantities of gases, water, and light (e.g., proportions, quantities, etc.).

For example, the photosynthesis optimization program 134 may determine that the cost of diverting the CO₂ from the other artificial photosynthesis unit involves a negligible cost. However, the cost of producing artificial sunlight for both artificial photosynthesis units will be substantial over the span of 9 hours required (10 am to 7 pm). Moreover, the cost-benefit analysis model indicates that artificial sunlight bulbs have an impermissibly high tendency to malfunction when operated for several hours continuously, which increases projected maintenance costs and reduces the net benefit. The photosynthesis optimization program 134 also determines that the illumination control system can be adjusted sub-ideally, with little cost, but that the resultant benefit is below a threshold. The photosynthesis optimization program 134 determines that transporting the artificial photosynthesis units to either the nearby stretch of highway or another factory involves an acceptable cost of fuel and labour. Although the satellite imaging and weather forecasts demonstrate that the ambient levels of CO₂, humidity, and sunlight are no longer ideal at the nearby stretch of highway—the other factory is considerably further away which entails significant cost at prevailing gas prices in the area retrieved via the network 108. Thus, the potential adjustments for the optimized reaction based on the cost-benefit analysis is to move the operation to the nearby stretch of highway and install the illumination control system to reflect concentrated light with minimal need for artificial sunlight. The photosynthesis optimization program 134 communicates to a user-operated computing device 120 on-site at the factory and coordinates the move which will require trucks and three other staffed individuals to assist in transporting the artificial photosynthesis units and the illumination control system. Once setup at the nearby stretch of highway, the illumination control system reflectors are automatically adjusted to maximize irradiance to the artificial photosynthesis units between 10 am and 5 pm, and between 5 pm and 7 pm, minimal artificial sunlight will be used.

In accordance with the exemplary embodiment of the present inventive concept illustrated with reference to FIG. 2C, the photosynthesis optimization program 134 may use the shown system architecture to perform photosynthesis optimization 200. The optimize control parameter inputs/outputs might not be limited to those shown. For example, cost-benefit may be an additional input.

FIG. 3 illustrates a block diagram depicting the hardware components of the photosynthesis optimization system 100 of FIG. 1, in accordance with an exemplary embodiment of the present inventive concept.

It should be appreciated that FIG. 3 provides only an illustration of one implementation and does not imply any limitations regarding the environments in which different embodiments may be implemented. Many modifications to the depicted environment may be made.

Devices used herein may include one or more processors 302, one or more computer-readable RAMs 304, one or more computer-readable ROMs 306, one or more computer readable storage media 308, device drivers 312, read/write drive or interface 314, network adapter or interface 316, all interconnected over a communications fabric 318. Communications fabric 318 may be implemented with any architecture designed for passing data and/or control information between processors (such as microprocessors, communications and network processors, etc.), system memory, peripheral devices, and any other hardware components within a system.

One or more operating systems 310, and one or more application programs 311 are stored on one or more of the computer readable storage media 308 for execution by one or more of the processors 302 via one or more of the respective RAMs 304 (which typically include cache memory). In the illustrated embodiment, each of the computer readable storage media 308 may be a magnetic disk storage device of an internal hard drive, CD-ROM, DVD, memory stick, magnetic tape, magnetic disk, optical disk, a semiconductor storage device such as RAM, ROM, EPROM, flash memory or any other computer-readable tangible storage device that can store a computer program and digital information.

Devices used herein may also include a R/W drive or interface 314 to read from and write to one or more portable computer readable storage media 326. Application programs 311 on said devices may be stored on one or more of the portable computer readable storage media 326, read via the respective R/W drive or interface 314 and loaded into the respective computer readable storage media 308.

Devices used herein may also include a network adapter or interface 316, such as a TCP/IP adapter card or wireless communication adapter (such as a 4G wireless communication adapter using OFDMA technology). Application programs 311 on said computing devices may be downloaded to the computing device from an external computer or external storage device via a network (for example, the Internet, a local area network or other wide area network or wireless network) and network adapter or interface 316. From the network adapter or interface 316, the programs may be loaded onto computer readable storage media 308. The network may comprise copper wires, optical fibers, wireless transmission, routers, firewalls, switches, gateway computers and/or edge servers.

Devices used herein may also include a display screen 320, a keyboard or keypad 322, and a computer mouse or touchpad 324. Device drivers 312 interface to display screen 320 for imaging, to keyboard or keypad 322, to computer mouse or touchpad 324, and/or to display screen 320 for pressure sensing of alphanumeric character entry and user selections. The device drivers 312, R/W drive or interface 314 and network adapter or interface 316 may comprise hardware and software (stored on computer readable storage media 308 and/or ROM 306).

The programs described herein are identified based upon the application for which they are implemented in a specific one of the exemplary embodiments. However, it should be appreciated that any particular program nomenclature herein is used merely for convenience, and thus the exemplary embodiments should not be limited to use solely in any specific application identified and/or implied by such nomenclature.

It is to be understood that although this disclosure includes a detailed description on cloud computing, implementation of the teachings recited herein are not limited to a cloud computing environment. Rather, the exemplary embodiments of the present inventive concept are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or data center).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer can deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

FIG. 4 illustrates a cloud computing environment, in accordance with an exemplary embodiment of the present inventive concept.

As shown, cloud computing environment 50 may include one or more cloud computing nodes 40 with which local computing devices used by cloud consumers, such as, for example, personal digital assistant (PDA) or cellular telephone 54A, desktop computer 54B, laptop computer 54C, and/or automobile computer system 54N may communicate. Nodes 40 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 4 are intended to be illustrative only and that computing nodes 40 and cloud computing environment 50 can communicate with any type of computerized device over any type of network and/or network addressable connection (e.g., using a web browser).

FIG. 5 illustrates abstraction model layers, in accordance with an exemplary embodiment of the present inventive concept.

Referring now to FIG. 5, a set of functional abstraction layers provided by cloud computing environment 50 (FIG. 4) is shown. It should be understood in advance that the components, layers, and functions shown in FIG. 5 are intended to be illustrative only and the exemplary embodiments are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfilment provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and photosynthesis optimization 96.

The exemplary embodiments of the present inventive concept may be a system, a method, and/or a computer program product at any possible technical detail level of integration. The computer program product may include a computer readable storage medium (or media) having computer readable program instructions thereon for causing a processor to carry out aspects of the present inventive concept.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present inventive concept may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present inventive concept.

Aspects of the present inventive concept are described herein with reference to flowchart illustrations and/or block diagrams of methods, apparatus (systems), and computer program products according to exemplary embodiments. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present inventive concept. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Based on the foregoing, a computer system, method, and computer program product have been disclosed. However, numerous modifications, additions, and substitutions can be made without deviating from the scope of the exemplary embodiments of the present inventive concept. Therefore, the exemplary embodiments of the present inventive concept have been disclosed by way of example and not by limitation.

1. A method for photosynthesis optimization, the method comprising:
determining ambient levels of at least one gas, water, and sunlight at a location;
selecting a catalyst to perform an artificial photosynthesis reaction at the location;
determining at least one limiting factor for the artificial photosynthesis reaction based on the catalyst and the ambient levels; and
compensating for the at least one limiting factor.

2. The method of claim 1,
wherein the determining ambient levels of the at least one gas, water, and sunlight at the location is performed by machine learning based on at least one of a weather forecast, previously recorded ambient levels of the at least one gas, water, and sunlight, and satellite imaging.

3. The method of claim 1,
wherein at least one of the location and the catalyst is chosen based on the ambient levels of the at least one gas, water, and sunlight.

4. The method of claim 1,
wherein the at least one gas includes carbon dioxide, and
wherein the ambient levels of carbon dioxide are determined using at least one of satellite imaging and carbon sequestration at the location.

5. The method of claim 1, further comprising:
performing a cost-benefit analysis of compensating for the at least one limiting factor; and
implementing a compensatory option from a plurality of compensatory options based on the cost-benefit-analysis.

6. The method of claim 5, wherein the compensatory option is performed by adjusting at least one of an IoT controlled solar irradiance mirror, an artificial light source, a gas inlet, and a humidity controller.

7. The method of claim 1,
wherein the catalyst is a photocatalyst, bio-electrochemical catalyst, or a photochemical catalyst, and
wherein the selecting the catalyst is based on a humidity, irradiance, and carbon dioxide sensitivity profile and knowledge base.

8. The method of claim 7, further comprising:
generating a three-dimensional lookup table for the catalyst based on the humidity, irradiance, and carbon dioxide sensitivity profile.

9. A computer program product for photosynthesis optimization, the computer program product comprising:
one or more non-transitory computer-readable storage media and program instructions stored on the one or more non-transitory computer-readable storage media capable of performing a method, the method comprising:
determining ambient levels of at least one gas, water, and sunlight at a location;
selecting a catalyst to perform an artificial photosynthesis reaction at the location;
determining at least one limiting factor for the artificial photosynthesis reaction based on the catalyst and the ambient levels; and
compensating for the at least one limiting factor.

10. The method of claim 9,
wherein the determining ambient levels of the at least one gas, water, and sunlight at the location is performed by machine learning based on at least one of a weather forecast, previously recorded ambient levels of the at least one gas, water, and sunlight, and satellite imaging.

11. The method of claim 9,
wherein at least one of the location and the catalyst is chosen based on the ambient levels of the at least one gas, water, and sunlight.

12. The method of claim 9,
wherein the at least one gas includes carbon dioxide, and
wherein the ambient levels of carbon dioxide are determined using at least one of satellite imaging and carbon sequestration at the location.

13. The method of claim 9, further comprising:
performing a cost-benefit analysis of compensating for the at least one limiting factor; and
implementing a compensatory option from a plurality of compensatory options based on the cost-benefit-analysis.

14. The method of claim 13,
wherein the compensatory option is performed by adjusting at least one of an IoT controlled solar irradiance mirror, an artificial light source, a gas inlet, and a humidity controller.

15. A computer system for photosynthesis optimization, the system comprising:
one or more computer processors, one or more computer-readable storage media, and program instructions stored on the one or more of the computer-readable storage media for execution by at least one of the one or more processors capable of performing a method, the method comprising:
determining ambient levels of at least one gas, water, and sunlight at a location;
selecting a catalyst to perform an artificial photosynthesis reaction at the location;
determining at least one limiting factor for the artificial photosynthesis reaction based on the catalyst and the ambient levels; and
compensating for the at least one limiting factor.

16. The method of claim 15,
wherein the determining ambient levels of the at least one gas, water, and sunlight at the location is performed by machine learning based on at least one of a weather forecast, previously recorded ambient levels of the at least one gas, water, and sunlight, and satellite imaging.

17. The method of claim 15,
wherein at least one of the location and the catalyst is chosen based on the ambient levels of the at least one gas, water, and sunlight.

18. The method of claim 15,
wherein the at least one gas includes carbon dioxide, and
wherein the ambient levels of carbon dioxide are determined using at least one of satellite imaging and carbon sequestration at the location.

19. The method of claim 15, further comprising:
performing a cost-benefit analysis of compensating for the at least one limiting factor; and
implementing a compensatory option from a plurality of compensatory options based on the cost-benefit-analysis.

20. The method of claim 19,
wherein the compensatory option is performed by adjusting at least one of an IoT controlled solar irradiance mirror, an artificial light source, a gas inlet, and a humidity controller.
